(19) United States
(12) Patent Application Publication
(10) Pub. No.: US 2024/0189236 A1
(43) Pub. Date: Jun. 13, 2024

(54) COMPOSITION COMPRISING A (METH)ACRYLATE COPOLYMER, AN ALKALI OR AMMONIUM SALT OF A SATURATED ALIPHATIC MONOCARBOXYLIC ACID AND SPECIFIC GLIDANTS

(71) Applicant: Evonik Operations GmbH, Essen (DE)

(72) Inventors: Erna ROTH, Darmstadt (DE); Andrea ENGEL, Birmingham, AL (US); Peter NIEPOTH, Groß-Umstadt (DE); Lisa GÜTTLER, Griesheim (DE); Andreas KLOSENDORF, Bad Schwalbach (DE)

(73) Assignee: Evonik Operations GmbH, Essen (DE)

(21) Appl. No.: 18/577,357

(22) PCT Filed: Jul. 7, 2022
(86) PCT No.: PCT/EP2022/068831

§ 371 Date: Jan. 8, 2024

(30) Foreign Application Priority Data
Jul. 9, 2021 (EP) 21184702.5

(51) Int. Cl. A61K 9/16; A61K 8/02; A61K 31/522

(52) U.S. Cl. CPC A61K 9/1635; A61K 8/0254; A61K 9/1611; A61K 9/1617; A61K 9/1652; A61K 9/1682; A61K 31/522; A61K 2800/10; A61K 2800/41; A61K 2800/624

(57) ABSTRACT

A composition suitable as coating or binding agent for pharmaceutically, nutraceutically or cosmetically active ingredients includes:
a) at least one (meth)acrylate copolymer a), including polymerized units of 5-25% by weight of methacrylic acid and 75-95% by weight of C₁₋₄-alkylesters of methacrylic acid and/or C₁₋₄-alkylesters of acrylic acid, and
b) 1-25% by weight, based on the total weight of at least one (meth)acrylate copolymer a), of an alkali or ammonium salt of a saturated aliphatic monocarboxylic acid having 10 to 30 carbon atoms,
and c) or d) or both c) and d)
c) 2-25% by weight, based on the total weight of a), of at least one compound selected from glyceryl tristearate, hydroxypropyl methylcellulose, glyceryl behenate, and glyceryl monostearate and/or
d) 25-90% by weight, based on the total weight of a), of at least one selected from talc, rice hulls in powder form, magnesium stearate, corn starch, and microcrystalline cellulose.

### FIELD OF THE INVENTION

The invention refers to a composition, suitable as coating or binding agent for pharmaceutically, nutraceutically or cosmetically active ingredients, comprising the following components:
a) at least one (meth)acrylate copolymer a), comprising polymerized units of 5 to 25% by weight of methacrylic acid and 75 to 95% by weight of C1- to C4-alkylesters of methacrylic acid and/or C1- to C4-alkylesters of acrylic acid, and
b) 1 to 25% by weight, based on the total weight of at least one (meth)acrylate copolymer a), of an alkali or ammonium salt of a saturated aliphatic monocarboxylic acid having 10 to 30 carbon atoms,
   and c) or d) or both c) and d)
c) 2 to 25% by weight, based on the total weight of a), of at least one compound selected from glyceryl tristearate, hydroxypropyl methylcellulose, glyceryl behenate, and glyceryl monostearate and/or
d) 25 to 90% by weight, based on the total weight of a), of at least one compound selected from talc, rice hulls in powder form, magnesium stearate, corn starch, and microcrystalline cellulose.

Furthermore, the present invention refers to an aqueous dispersion, comprising water and 5 to 50% by weight of the composition according to the present invention as well as a dosage form, comprising a pharmaceutically, nutraceutically or cosmetically active ingredient and a polymeric coating or a polymeric matrix, wherein the polymeric coating or the polymeric matrix comprises the composition according to the present invention. Finally, the present invention pertains to the use of a composition according to the present invention as a coating or binding agent for pharmaceutically, nutraceutically or cosmetically active ingredients.

### BACKGROUND

Some health ingredients and API's require a protection from the acidic environment in the stomach or should release in the small intestine to avoid irritation of the mucosa or a burping effect. Therefore, an enteric coating is required to ensure a save release in the small intestine. There are certain polymers available in the market which show a delayed release for the supplements and pharmaceuticals.

For supplements, existing solutions may contain a combination of polymers such as cellulose and alginate or shellac and alginate, but there is no single polymer available which could give the desired properties.

EUDRAGIT® L 100 and EUDRAGIT® L 100-55 are well-known commercially available (meth)acrylate copolymer products for pharmaceutical applications. EUDRAGIT® L 100 is a copolymer polymerized from 50% by weight of methyl methacrylate and 50% by weight of methacrylic acid. The pH of the start of the specific active ingredient release in intestinal juice or simulated intestinal fluid is about pH 6.0. EUDRAGIT® L 100-55 is a copolymer polymerized from 50% by weight of ethyl acrylate and 50% by weight of methacrylic acid. The pH of the start of the specific active ingredient release in intestinal juice or simulated intestinal fluid is about pH 5.5.

However, EUDRAGIT® L 100 and EUDRAGIT® L 100-55 are not regulatory approved for supplements. Furthermore, there is a trend also for pharmaceuticals to reduce the total amount of carboxylic groups in a coating formulation or in a polymeric matrix formation with release at pH 6.8.

EUDRAGIT® FS is a copolymer polymerized from 10% by weight of methacrylic acid, 65% by weight of methyl acrylate, and 25% by weight of methyl methacrylate which the content of methacrylic acid groups is five times lower than that in EUDRAGIT® L 100 or EUDRAGIT® L 100-55. However, the pH at the start of the specific active ingredient release of the EUDRAGIT® FS polymer is above pH 7.0.

U.S. Pat. No. 5,644,011 discloses a coating agent comprising acrylic copolymer. The example 4 discloses that biscodyl pellets were coated with a mixture of acrylate polymer and glycerine monostearate. However, the said coating realesed the bisacodyl from the pellets at very faster rate at pH 6.8 Within 45 minutes 100% release).

Similarly, WO 2020/114714 A1 discloses that diprophylline pellets were coated with Eudragit FS 30D polymer. However, the active ingredient were released with 20 minutes of reaching a pH of 6.8. Eudragit FS is a copolymer polymerized from methyl methacrylate, methyl acrylate and methacrylic acid.

Thus, there is a need for enteric coating or binding compositions, which are easily dispersible in water to form dispersions suitable for creating enteric coatings on active ingredient containing cores or active ingredient containing matrix compositions. Further, there is a need of compositions and derived dosage forms with a comparably reduced content of acid groups which at the same time however would feature the start of release of active components in intestinal juice around below pH 7.0, for instance at pH 6.8. Thus, the objective of the presently claimed invention is to provide a coating which efficiently release active ingredient in intestine, at least 70%, preferably 80%, more preferably 90% of active ingredient after 180 mins, preferably 90 minutes at the target PH level 6.8.

The inventors of the present invention have surprisingly found that these objects can be solved by the specific composition according to the present invention, which requires at least one specific (meth)acrylate copolymer, at least one specific alkali or ammonium salt of a saturated aliphatic monocarboxylic acid in a specific amount as well as specific glidants in specific amounts. In particular, that only certain combinations of these three components in certain amounts lead to a desirable release at pH value 6.8.

### SUMMARY OF THE INVENTION

The present invention refers in a first aspect to a composition, suitable as coating or binding agent for pharmaceutically, nutraceutically or cosmetically active ingredients, comprising the following components:
a) at least one (meth)acrylate copolymer a), comprising polymerized units of 5 to 25% by weight of methacrylic acid and 75 to 95% by weight of C1- to C4-alkylesters of methacrylic acid and/or C1- to C4-alkylesters of acrylic acid, and
b) 1 to 25, preferably 5 to 18, % by weight, based on the total weight of at least one (meth)acrylate copolymer a), of an alkali or ammonium salt of a saturated aliphatic monocarboxylic acid having 10 to 30 carbon atoms,
and c) or d) or both c) and d)
c) 2 to 25, preferably 2 to 20, more preferably 5 to 15, % by weight, based on the total weight of a), of at least one compound selected from glyceryl tristearate, hydroxypropyl methylcellulose, glyceryl behenate, and glyceryl monostearate and/or
d) 25 to 90, preferably 25 to 75, more preferably 40 to 60, % by weight, based on the total weight of a), of at least one compound selected from talc, rice hulls in powder form, magnesium stearate, corn starch, and microcrystalline cellulose.

Furthermore, in a second aspect, the present invention pertains to an aqueous dispersion, comprising water and 5 to 50% by weight of the composition according to the present invention.

In a third aspect the present invention refers to a dosage form, comprising a pharmaceutically, nutraceutically or cosmetically active ingredient and a polymeric coating or a polymeric matrix, wherein the polymeric coating or the polymeric matrix comprises the composition according to present invention.

In a fourth aspect the present invention pertains to the use of a composition according to the present invention as a coating or binding agent for pharmaceutically, nutraceutically or cosmetically active ingredients.

Disclosed is a process for preparing the (meth)acrylate copolymer a) in the form of polymeric particles, comprising polymerized units of methacrylic acid and further monomers, with an overall monomer composition comprising polymerized units of 5 to 25% by weight of methacrylic acid and 75 to 95% by weight of further monomers, wherein the further monomers are selected from C1- to C4-alkylesters of methacrylic acid and/or C1- to C4-alkylesters of acrylic acid, by gradient emulsion polymerization, wherein the ratio by weight of polymerized units of methacrylic acid to further monomers is increasing in a gradient from the center to the surface of the particles and wherein the polymeric particles are obtained in the form of an aqueous dispersion.

The term “from the center to the surface of the particles” shall mean, assuming a round respectively a spherical particle, a direct way from the midpoint inside the polymeric particle (center) to (towards) the outside (surface) of the particle. The content of polymerized units of methacrylic acid increases from the center to the surface of the polymeric particle.

The particles of the (meth)acrylate copolymer a) polymer resulting from the disclosed process are deemed by the inventors to show an increased concentration of the carboxylic groups of the polymerized units of methacrylic acid on their surface compared to their overall low methacrylic acid content. Although the overall methacrylic acid content is comparatively low, it seems that the polymer particles as disclosed, when used as a coating or binding material in dosage forms comprising an active ingredient, act like copolymers or copolymer particles with much higher content of methacrylic acid. Thus, a process for preparing polymer particles with comparatively low allover methacrylic acid content and an unexpected low dissolution and active ingredient release behavior at the same time is provided.

It has been surprisingly found by the present inventors that a composition comprising the (meth)acrylate copolymer a) in combination with an alkali or ammonium salt of a saturated aliphatic monocarboxylic acid having 10 to 30 carbon atoms and the specific component c) and/or d) shows a release of the active ingredients even at lower pH than a composition comprising the (meth)acrylate copolymer a) alone. The composition according to the invention is suitable as coating or binding agent for pharmaceutically, nutraceutically or cosmetically active ingredients.

### DETAILED DESCRIPTION OF THE INVENTION

The present invention refers to a composition, suitable as coating or binding agent for pharmaceutically, nutraceutically or cosmetically active ingredients, comprising the following components:
a) at least one (meth)acrylate copolymer a), comprising polymerized units of 5 to 25% by weight of methacrylic acid and 75 to 95% by weight of C1- to C4-alkylesters of methacrylic acid and/or C1- to C4-alkylesters of acrylic acid, and
b) 1 to 25, preferably 5 to 18, % by weight, based on the total weight of at least one (meth)acrylate copolymer a), of an alkali or ammonium salt of a saturated aliphatic monocarboxylic acid having 10 to 30 carbon atoms,
and c) or d) or both c) and d)
c) 2 to 25, preferably 2 to 20, more preferably 5 to 15, % by weight, based on the total weight of a), of at least one compound selected from glyceryl tristearate, hydroxypropyl methylcellulose, glyceryl behenate, and glyceryl monostearate and/or
d) 25 to 90, preferably 25 to 75, more preferably 40 to 60, % by weight, based on the total weight of a), of at least one compound selected from talc, rice hulls in powder form, magnesium stearate, corn starch, and microcrystalline cellulose.

Preferably the solid content of the (meth)acrylate copolymer a) in the composition is at least 10, preferably 20 to 90% by weight.

### Component a): The (Meth)Acrylate Copolymer

The composition comprises at least one (meth)acrylate copolymer a).

The (meth)acrylate copolymer a) comprises polymerized units of 5 to 25% by weight of methacrylic acid and 75 to 95% by weight of C1- to C4-alkylesters of methacrylic acid and/or C1- to C4-alkylesters of acrylic acid. The (meth)acrylate copolymer a) may be present in the form of polymeric particles. Preferably the monomers may add up to 100%.

C1- to C4-alkyl esters of acrylic or methacrylic acid are in particular methyl methacrylate, ethyl methacrylate, butyl methacrylate, methyl acrylate, ethyl acrylate and butyl acrylate.

The (meth)acrylate copolymer a) may originate from an emulsion polymerization process, wherein the whole amount of the total monomers is charged and are polymerized simultaneously in one step to polymeric particles. As a result of the process, the polymeric particles show a unique distribution of the monomers, especially the polymerized units of methacrylic acid, can be deemed constant, from the center (inside) and surface (outside) of the particles.

The polymeric particles may be present in aqueous dispersed form or in the form of a re-dispersible powder gained by drying from an aqueous dispersion comprising the polymeric particles.

Preferably the (meth)acrylate copolymer a) comprises a monomer composition comprising polymerized units of 10 to 30% by weight of methyl methacrylate, 50 to 70% by weight of methyl acrylate and 5 to 15% by weight of methacrylic acid. Preferably the monomers preferably add up to 100%.

A suitable (meth)acrylate copolymer a) is the commercially available (meth)acrylate copolymer sold under the tradenames EUDRAGIT® FS 30 D or EUDRAGUARD® biotic, in the form of a 30% by weight aqueous dispersion. The copolymer is polymerized from 10% by weight of methacrylic acid, 65% by weight of methyl acrylate, and 25% by weight of methyl methacrylate.

The specific dissolution pH-value of the (meth)acrylate copolymer a), especially of the EUDRAGIT® FS 30 D polymer, is from about pH 7.0 to pH 7.2. There is no considerable dissolution below pH 7.0, for instance at pH 6.8.

### Process for Preparing a (Meth)Acrylate Copolymer a)

The (meth)acrylate copolymer a) may be prepared in a manner known in the art by free-radical polymerization of the monomers as described, for example, in EP 0 704 207 A2 and EP 0 704 208 A2. The (meth)acrylate copolymer a) may be prepared by conventional processes of free-radical polymerization continuously by batch processes, for example by emulsion polymerization in the presence of free-radical forming initiators and, where appropriate, regulators to adjust the molecular weight undiluted, in solution, by bead polymerization or in emulsion. The average molecular weight Mw (weight average, determined for example by measuring the solution viscosity) may be for example in the range from 80 000 g/mol to 1 000 000 g/mol.

Emulsion polymerization in aqueous phase in the presence of water-soluble initiators and, preferably anionic, emulsifiers is preferred. The weight-average size (radius) of the resulting polymeric particles is usually in the range from 50 to 500, preferably 80 to 300 nm, thus ensuring a viscosity below 1000 mPa's, which is favorable for processing techniques. The particle size can be determined by laser diffraction, e.g. using the Mastersizer 2000 (from Malvern Inc.).

In the case of bulk polymerization, the copolymer can be obtained in solid form by crushing, extrusion, granulation or hot cut.

The (meth)acrylate copolymer a) can be obtained in a manner known in the art by free-radical bulk, solution, bead or emulsion polymerization. It can be brought before processing to the appropriate particle size range by suitable grinding, drying or spraying processes. This can be achieved by simple crushing of extruded and cooled pellets or hot cut. The use of polymer powder may be advantageous, especially for mixing with other powders or liquids. Typical equipment suitable for producing of powders is well known to those skilled in the art, e.g. air jet mill, pinned disc mill, compartment mill. It is possible, where appropriate to include appropriate sieving steps. A suitable mill for industrial large quantities is, for example, an opposed jet mill (Multi No. 4200) operated with a gauge pressure of about 6 bar.

An emulsion polymerization process may advantageously be carried out by the monomer emulsion feed process or the monomer feed process, respectively, in a polymerization reactor. For this, water is heated to the reaction temperature in a polymerization reactor. Surfactants and/or initiators may be added at this stage. The whole amounts of all monomers may be charged into the reactor before adding the initiator. This method is often referred to as “batch emulsion process”.

Emulsifiers which may be used are especially anionic and non-ionic surfactants. The amount of emulsifier used is generally not more than 5% by weight, preferably 0.1 to 3% by weight, based on the total weight of the monomers. Typical emulsifiers are for example alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and others.

Beside those polymerization initiators conventionally used in emulsion polymerization (e.g. per-compounds, such as ammonium peroxodisulfate (APS)) redox systems, such as sodium disulphite-APS-iron, can be applied. Also, water-soluble azo initiators may be applied and/or a mixture of initiators can be used. The amount of initiator is usually between 0.005 to 0.5, preferably 0.01 to 0.3% by weight, based on the weight of the monomers.

A chain transfer agent may be added to improve the process stability and the reproducibility of the weight average molecular weight (Mw). A typical amount of chain transfer agent may be 0.05 to 1% by weight based on monomer weight. A typical chain transfer agent may be, for example, thioglycolic acid 2-ethyl hexyl ester (TGEH) or n-dodecyl mercaptan (nDDM). However, the chain transfer agent may be omitted in some cases, without affecting the properties according to the invention.

A typical emulsion polymerization broth may comprise the monomers and water at a typical ratio by weight of about 3 to 7 as main components and 0.005 to 0.5% by weight of one more polymerization initiator(s), 0.05 to 1% by weight of a chain transfer agent(s), less than 5% by weight or 0.1 to 3.0% by weight of an emulsifier and 0 to 0.5% by weight of an antifoam agent, wherein all components preferably add up to 100%.

The polymerization temperature depends on the initiators within certain limits. For example, if APS is used, it is advantageous to operate in the range from 60 to 90° C.; if redox systems are used it is also possible to polymerize at lower temperatures, for example at 30° C.

At the end of the process the reactor content is usually allowed to cool down, for instance to 20 to 25° C. and the resulting dispersion may be filtered, for instance through a 250 μm gaze.

The average particle size (D50) of the polymeric particles produced in the emulsion polymerization can range from 50 to 500, preferably 80 to 300 nm. The average particle size of the polymer particles may be determined by methods well known to a skilled person, for instance by the method of laser diffraction. The particle size may be determined by laser diffraction, using a Mastersizer 2000 (Malvern). The values can be indicated as particle radius rMS [nm], which is half of the median of the volume-based particle size distribution d(v,50).

The dispersion can also be dried to a powder or granulate, preferably by spray drying, spray granulation, freeze drying, coagulation or extrusion. Thus, a solid powder or granulate can be obtained, which offers certain advantages with regard to handling and logistics. The dry powder or granulate may be used as polymeric binder for matrix dosage forms.

The dried polymerizate may then be transferred into a coating suspension by re-dispersing the solid in water, e.g. (where required) by the use of a high shear mixer.

### Aqueous Dispersion of the (Meth)Acrylate Copolymer a)

The (meth)acrylate copolymer a) is usually gained from an emulsion polymerization process in the form of an aqueous dispersion or commercially available as such a dispersion (EUDRAGIT® FS 30 D), for instance at a polymer concentration of about 30% by weight. The components b) and c) and/or d)) and optionally pharmaceutical, nutraceutical or cosmetic excipients may then be added to the aqueous dispersion for further processing in applications as coating or binding agent.

### Powder or Granulate

The (meth)acrylate copolymer a) may be converted from an aqueous dispersion to a dry form, preferably to a powder or a granulate, by spray drying, spray granulation, spray agglomeration, freeze drying, coagulation or extrusion of the aqueous dispersion. The resulting granulate or powder may have a particle size D50 in the range from about 0.01 to 5 mm. Powder may have a particle size D50 in the range from about 0.01 up to less than 0.5 mm. Granulates may have a particle size D50 in the range from about 0.5 mm up to 5 mm. The average particle size of granulates is preferably determined by well-known sieving methods. The particle size D50 of powder is preferably determined by laser diffraction. The dry form of (meth)acrylate copolymer a) may be used for re-dispersion to an aqueous dispersion or alternatively for dry mixing with the components b) and c) and/or d)) to gain a (ready to use) composition in dry form as disclosed. The dry form may be converted again to an aqueous dispersion, optionally pharmaceutical, nutraceutical or cosmetic excipients may then be added for further processing in applications as coating or binding agent.

### Component b): Monocarboxylic Acid

The composition comprises 1 to 25, preferably 5 to 15% by weight, based on the total weight of the at least one (meth)acrylate copolymer a), of an alkali or ammonium salts of a saturated aliphatic monocarboxylic acid having 10 to 30 carbon atoms.

The alkali or ammonium salt of the saturated aliphatic monocarboxylic acid having 10 to 30 carbon atoms may be selected from alkali or ammonium salts of the following monocarboxylic acids: decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid, and triacontanoic acid (melissic acid, C30).

Preferably, the alkali salt of a saturated aliphatic monocarboxylic acid having 10 to 30 carbon atoms is sodium stearate.

### Components c) and d)

The composition comprises, in addition to the components a) and b), the components c) or d) or (both) c) and d).

### Components c): Glyceryl Tristearate, Hydroxypropyl Methylcellulose, Glyceryl Behenate, and Glyceryl Monostearate

The composition can comprise 2 to 25, preferably 2 to 20, more preferably 5 to 15, % by weight, based on the total weight of at least one (meth)acrylate copolymer a), of at least one compound selected from glyceryl tristearate, hydroxypropyl methylcellulose, glyceryl behenate, and glyceryl monostearate or mixtures thereof. In one embodiment the compounds is glyceryl tristearate. In one embodiment the compound is hydroxypropyl methylcellulose. In one embodiment the compound is glyceryl behenate. In one embodiment the compound is glyceryl monostearate.

### Components d): Talc, Rice Hulls in Powder Form, Magnesium Stearate, Corn Starch, and Microcrystalline Cellulose

The composition may comprise 25 to 90, preferably 25 to 75, more preferably 40 to 60, % by weight, based on the total weight of at least one (meth)acrylate copolymer a), at least one compound selected from talc, rice hulls in powder form, magnesium stearate, corn starch, and microcrystalline cellulose or mixtures thereof. In one embodiment the compound is talc. In one embodiment the compound is rice hulls in powder form, for example commercially available under the tradename Nu-FLOW® from RIBUS Inc or Nu-MAG® from RIBUS Inc. In one embodiment the compound is magnesium stearate. In one embodiment the compound is corn starch. In one embodiment the compound is microcrystalline cellulose.

### Preparation of the Composition

The composition as disclosed may be prepared in many ways by mixing the components a), b) and c) and/or d). The following example may give a guideline: The intended amount of the alkali or ammonium salts of a saturated aliphatic monocarboxylic acid having 10 to 30 carbon atoms b), for instance sodium stearate, may be dissolved or dispersed at a concentration of about 1 to 10% by weight in water by heating to about 50 to 80° C. The resulting solution or dispersion is then added to about roughly the same volume of a dispersion of the at least one (meth)acrylate copolymer a) of about 30% by weight polymer content, for instance by use of EUDRAGIT® FS 30 D to give an intermediate dispersion. The intermediate dispersion may show a (meth)acrylate copolymer a) content of about 12 to 18% by weight. Then components c) and/or d) may be added to the resulting dispersion under vigorous stirring, for instance for 2 to 20 min at 10.000 to 15.000 rpm. The resulting final dispersion may be dried in a spray drying process, for instance at an inlet temperature of about 75 to 85° C. and an outlet temperature of about 45 to 50° C. The resulting powder product comprises the composition as disclosed and may be directly processed or alternatively used in re-dispersed form as a coating or binding agent for active ingredient containing dosage forms. Alternatively, the resulting final dispersion is directly used without previously drying.

### Dosage Form

The dosage form according to the present invention comprises the composition according to the present invention, a pharmaceutically, nutraceutically or cosmetically active ingredient and a polymeric coating or a polymeric matrix, wherein the polymeric coating or the polymeric matrix comprises the composition according to the present invention.

A polymeric coating may be applied, for instance, by spray coating of an aqueous dispersion comprising the at least one (meth)acrylate copolymer a) polymeric particles with the at least one components b) and c) and/or d) and optionally pharmaceutical, nutraceutical or cosmetic excipients added, onto a core comprising a pharmaceutical, nutraceutical or cosmetic active ingredient. The amount of the coating layer may be in the range of about 3 to 50, preferably 4 to 30, % by weight based on the weight of the core. The thickness of the coating layer may be in the range of about 10 to 100, preferably 15 to 80, μm.

A polymeric matrix may be derived, for instance, from an aqueous dispersion comprising the composition of the present invention and optionally pharmaceutical, nutraceutical or cosmetic excipients added, or from a spray dried powder of such an aqueous dispersion, by methods such as wet or dry granulation, extrusion granulation or powder binding with the addition a pharmaceutically, nutraceutically or cosmetically active ingredient and optionally additional pharmaceutical, nutraceutical or cosmetic excipients, such as antioxidants, brighteners, binding agents, flavouring agents, flow aids, fragrances, penetration-promoting agents, pigments, plasticizers, polymers, pore-forming agents or stabilizers.

The dosage form may be a coated dosage form comprising a core, comprising an active ingredient, preferably a nutraceutically active ingredient and a polymer coating onto the core, wherein the coating comprises a polymer film derived from the aggregation of the polymeric particles during the film forming process. The dosage form may be, for instance, in the form of a coated or uncoated pellet, a coated or uncoated tablet, a capsule filled with pellets, a sachet and so on.

The dosage form may be a matrix dosage form comprising an active ingredient, preferably a nutraceutically active ingredient, embedded in a polymeric matrix derived from the aggregation of the polymeric particles during the matrix forming process.

### Active Ingredient Release

A dosage form with a pharmaceutically, nutraceutically or cosmetically active ingredient containing core, coated with the composition according to the present invention, may show an active ingredient release of 10% or less after 1 or 2 hours at pH 1.2 (in media according to USP 41 method 2, Paddle 100 rpm).

A dosage form with a pharmaceutically, nutraceutically or cosmetically active ingredient containing core, coated with the composition as disclosed, may show with an active ingredient release of 10% or less after 2 hours in pH 1.2 medium and of 40% or more, preferably 42% or more in subsequent pH 6.8 medium after 60 min (media are according to USP 41 method 2, Paddle 100 rpm).

A dosage form with a pharmaceutically, nutraceutically or cosmetically active ingredient containing core, coated with the composition as disclosed, may show an active ingredient release of 10% or less after 2 hours in pH 1.2 medium and of 70% or more, preferably 80% or more in subsequent pH 6.8 medium after 90 min (media are according to USP 41 method 2, Paddle 100 rpm).

### Pharmaceutically, Nutraceutically or Cosmetically Active Ingredients

Pharmaceutical, nutraceutical or cosmetical active ingredients may be selected from pre-biotics and from pro-biotics. In one embodiment the ingredients are selected from substances, which can create burping, irritate the mucosa or are sensitive towards the acid environment in the stomach e.g. fish oil, garlic oil, chili extract, vitamins, and enzymes.

Pharmaceutically and also nutraceutically or cosmetically active ingredients can be selected from analgetics, antibiotics or anti-infectives, antibodies, antiepileptics, antigens from plants, antirheumatics, betablocker, benzimidazole derivatives, beta-blocker, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptidhormones proteins, therapeutical bacteria, peptides, urology drugs and vaccines.

In one embodiment the pharmaceutically, nutraceutically or cosmetically active ingredients is selected from fish oil, garlic oil, chili extract, vitamins, enzymes, minerals, green tea, herbals, essential oils, hemp, probiotics, prebiotics, fiber, amino acids, eucalyptus oil, orange oil, haarlem oil, ginseng, ginger, fungus, phosphatidylcholine, natural extracts, glucosamine, chondroitin, lipoid acid or mixtures thereof.

### Pharmaceutically Active Ingredients

The invention is preferably useful for pharmaceutically active ingredients where the total amount of carboxylic groups in the coating formulation or in the polymeric matrix formation shall be kept low but the active ingredient release is intended to start already in the range of pH 4.3 to 5.8.

The therapeutical and chemical classes of pharmaceutical (and also nutraceutically or cosmetically) active ingredients used in the dosage forms as disclosed may be selected from analgetics, antibiotics or anti-infectives, antibodies, antiepileptics, antigens from plants, antirheumatics, betablocker, benzimidazole derivatives, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptide hormone proteins, therapeutical bacteria, peptides, urology drugs and vaccines.

Examples of pharmaceutical active ingredients may be: acamprosat, aescin, amylase, acetylsalicylic acid, adrenalin, 5-amino salicylic acid, aureomycin, bacitracin, balsalazine, beta carotene, bicalutamid bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexlansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormone (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, methylphenidate, milameline, minerals, minoprazole, naproxen, natamycin, nitrofurantion, novobiocin, olsalazine, omeprazole, orothates, pancreatin, pantoprazole, parathyroid hormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or one or more kinds of mixtures or combinations thereof. A suitable pharmaceutically active ingredient is, for instance, diprophylline.

Nutraceutically and/or Cosmetically Active Ingredients

Nutraceuticals and/or cosmetics are well known to the skilled person. Nutraceuticals and cosmetics are often defined as extracts of foods claimed to have medical effects on human health. Thus, nutraceutically and/or cosmetically active ingredients may display pharmaceutical activities as well: Examples for nutraceutically active ingredients may be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus, it is clear that more or more substances listed as nutraceuticals or cosmetics may also be indicated as pharmaceutical active ingredients.

Depending on the territory, the specific application, the local authority legislation and classification, the same substance may be listed as a pharmaceutically or as a nutraceutically or cosmetically active ingredient respectively as a pharmaceutical or a nutraceutical or cosmetic composition or even both. Thus, it is evident to a skilled person that there is a broad overlap between the terms pharmaceutically, nutraceutically and/or cosmetically active ingredients respectively and pharmaceutically or nutraceutically or cosmetically compositions.

Nutraceuticals, cosmetics or nutraceutically and cosmetically active ingredients are sometimes defined as extracts of foods claimed to have medical effects on human health.

Nutraceuticals, cosmetics or nutraceutically and cosmetically active ingredients may also include probiotics and prebiotics. Probiotics are living microorganisms believed to support human or animal health when consumed, for example certain strains of the genera Lactobacillus or Bifidobacterium. Prebiotics are nutraceuticals or nutraceutical active ingredients that induce or promote the growth or activity of beneficial microorganisms in the human or animal intestine.

The nutraceutically and cosmetically active ingredient may be usually contained in a medical form such as capsule, tablet or powder in a prescribed dose. Examples for nutraceuticals or cosmetics are resveratrol from grape products or (pro-)anthocyanines from berries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or anthocyanins from berries. Sometimes the expression neutraceuticals or nutriceuticals are used as synonyms for nutraceuticals.

### Pharmaceutically, Nutraceutically or Cosmetically Excipients

Pharmaceutically, nutraceutically or cosmetically excipients are excipients well known to the skilled person and widely used in pharmacy, nutraceutics and cosmetics. The pharmaceutically, nutraceutically or cosmetically excipients are optional excipients and are different from the essential components a), b), and c) and/or d) according to the invention.

The composition can further comprise 0 to 400 or 0.1 to 400, preferably 0 to 200 or 0.1 to 200, most preferably 10 to 100% by weight, based on the total weight of the at least one (meth)acrylate copolymer a), of additional pharmaceutically, nutraceutically or cosmetically excipients.

Preferably the additional pharmaceutically, nutraceutically or cosmetically excipients are selected from the classes of antioxidants, brighteners, flavouring agents, flow aids, fragrances, penetration-promoting agents, pigments, plasticizers, polymers, pore-forming agents or stabilizers or combinations thereof.

The term pharmaceutical, nutraceutical or cosmetic excipient is well known to the skilled person. Excipients are customary used in pharmacy but also in the field of nutraceuticals or cosmetics, occasionally they are also referred as customary additives. It is, of course, always necessary for all the excipients or customary additives employed to be toxicologically acceptable and usable in particular in medicaments, nutraceutics or cosmetics without a risk for customers or patients.

Although the requirements are usually higher in the pharmaceutical field there is a wide overlap of excipients used for pharmaceutical purposes and those used for nutraceutical or cosmetic purposes. Usually all pharmaceutical excipients may be used for nutraceutical or cosmetic purposes and at least a large number of nutraceutical excipients are allowed to be used for pharmaceutical purposes as well. Excipients may be added to the formulation of the invention, preferably as admixtures to suspensions for spray coating.

### Use

Described is the use of the composition as disclosed as a coating or binding agent for pharmaceutically, nutraceutically or cosmetically active ingredients (A composition for use as coating or binding agent for pharmaceutically, nutraceutically or cosmetically active ingredients).

### Plasticizers

The composition may comprise 2 to 40% by weight of a plasticizer, based on the total weight of the at least one (meth)acrylate copolymer a). Less than 2% by weight of a plasticizer, based on the total weight of the at least one (meth)acrylate copolymer a) or no plasticizer at all may be also comprised.

Plasticizers may be defined in that they achieve through physical interaction with a polymer a reduction in the glass transition temperature and promote film formation, depending on the added amount. Suitable substances usually have a molecular weight of between 100 and 20,000 and comprise one or more hydrophilic groups in the molecule, e.g. hydroxy ester or amino groups.

The plasticizer may be selected from the groups of alkyl citrates, glycerol esters, alkyl phthalates, alkyl sebacates, sucrose esters, sorbitan esters and polyethylene glycols.

The plasticizer may be selected from triethyl citrate (TEC), acetyl triethyl citrate (ATEC), diethyl sebacate and dibutyl sebacate (DBS), glycerol, propylene glycol, polyethylene glycols 200 to 12,000 and castor oil.

### Re-Dispersible Powder

The composition may be present in the form of an aqueous dispersion and may then be spray dried to give a re-dispersible powder.

### Aqueous Dispersion

The composition may be present in the form of an aqueous dispersion, comprising water and 5 to 50, preferably 8 to 40, more preferably 8 to 20, % by weight of the composition.

### Typical Composition

A typical composition may comprise:
a) the at least one (meth)acrylate copolymer a);
b) 1 to 25% by weight, based on the total weight of a), of sodium stearate,
and c) or d) or both
c) 5 to 15% by weight, based on a), hydroxypropyl methylcellulose and/or glyceryl tristearate;
d) 40 to 60% by weight, based on a), of talc and/or magnesium stearate;
wherein the components a), b), c) and d) add up to 100% dry weight.

### Items

The invention may be characterized by the following items:

1. Composition, suitable as coating or binding agent for pharmaceutically, nutraceutically or cosmetically active ingredients, comprising the following components, preferably in solid, dissolved or in dispersed form:
a) at least one (meth)acrylate copolymer a), comprising polymerized units of 5 to 25% by weight of methacrylic acid and 75 to 95% by weight of C1- to C4-alkylesters of methacrylic acid and/or C1- to C4-alkylesters of acrylic acid, and
b) 1 to 25, preferably 5 to 18, % by weight, based on the total weight of at least one (meth)acrylate copolymer a), of an alkali or ammonium salt of a saturated aliphatic monocarboxylic acid having 10 to 30 carbon atoms,
and c) or d) or both c) and d)
c) 2 to 25, preferably 2 to 20, more preferably 5 to 15, % by weight, based on the total weight of a), of at least one compound selected from glyceryl tristearate, hydroxypropyl methylcellulose, glyceryl behenate, and glyceryl monostearate and/or
d) 25 to 90, preferably 25 to 75, more preferably 40 to 60, % by weight, based on the total weight of a), of at least one compound selected from talc, rice hulls in powder form, magnesium stearate, corn starch, and microcrystalline cellulose.

2. Composition according to item 1, wherein the (meth)acrylate copolymer a) comprises an overall monomer composition by weight comprising polymerized units of 10 to 30% by weight of methyl methacrylate, 50 to 70% by weight of methyl acrylate and 5 to 15% by weight of methacrylic acid.

3. Composition according to item 1 or 2, wherein the alkali or ammonium salt of a saturated aliphatic monocarboxylic acid having 10 to 30 carbon atoms is selected from alkali or ammonium salts of decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid and triacontanoic acid (melissic acid, C30).

4. Composition according to any of the preceding items, wherein the alkali salt of the saturated aliphatic monocarboxylic acid is sodium stearate.

5. Composition according to any of the preceding items, wherein up to 400% by weight, based on the total weight of the at least one (meth)acrylate copolymer a), of additional pharmaceutical, nutraceutical or cosmetic excipients are comprised.

6. Composition according to any of the preceding items, wherein up to 200% by weight, based on the total weight of the at least one (meth)acrylate copolymer a), of additional pharmaceutical, nutraceutical or cosmetic excipients are comprised.

7. Composition according to item 5 or 6, wherein pharmaceutical, nutraceutical or cosmetic excipients are comprised, which are selected from the classes of antioxidants, brighteners, flavouring agents, flow aids, fragrances, penetration-promoting agents, pigments, plasticizers, polymers, pore-forming agents or stabilizers or combinations thereof.

8. Composition according to any of the preceding items, wherein a plasticizer is present in an amount of 2 to 40% by weight, based on the total weight of the at least one (meth)acrylate copolymer a).

9. Composition according to any of the preceding items, wherein a plasticizer is present which is selected from the groups of alkyl citrates, glycerol esters, alkyl phthalates, alkyl sebacates, sucrose esters, sorbitan esters and polyethylene glycols.

10. Composition according to any of the preceding items, wherein a plasticizer is present which is selected from triethyl citrate (TEC), acetyl triethyl citrate (ATEC), diethyl sebacate and dibutyl sebacate (DBS), glycerol, propylene glycol, polyethylene glycols with an number average molecular weight of 200 to 12,000 g/mol, preferably measured with GPC employing polystyrene standards, and castor oil.

11. Composition according to any of the preceding items, in the form of an aqueous dispersion.

12. Composition according to any of the preceding items, in the form of a re-dispersible powder.

13. Composition according to any of the preceding items, comprising
a) the at least one (meth)acrylate copolymer a);
b) 1 to 25% by weight, based on the total weight of a), of sodium stearate, and c) or d) or both
c) 5 to 15% by weight, based on a), hydroxypropyl methylcellulose and/or glyceryl tristearate;
d) 40 to 60% by weight, based on a), of talc and/or magnesium stearate;
wherein the components a), b), c) and d) add up to 100% dry weight.

14. Composition according to any of the preceding items, wherein the solid content of the at least one (meth)acrylate copolymer a) in the composition is at least 5, preferably 20 to 90, % by weight, based on the total weight of the composition.

15. Aqueous dispersion, comprising water and 5 to 50% by weight of the composition according to any of the preceding items.

16. Dosage form, comprising a pharmaceutically, nutraceutically or cosmetically active ingredient and a polymeric coating or a polymeric matrix, wherein the polymeric coating or the polymeric matrix comprises the composition according to any of the preceding items.

17. Dosage form according to item 16, comprising a pharmaceutically, nutraceutically or cosmetically active ingredient containing core, coated with the composition according to any of the preceding items, and with an active ingredient release of 10% or less after 1, preferably 2, hours at pH 1.2.

18. Dosage form according to item 16 or 17, comprising a pharmaceutically, nutraceutically or cosmetically active ingredient containing core, coated with the composition according to any one of items 1 to 14 and with an active ingredient release of 10% or less after 2 hours in pH 1.2 medium and of 40% or more, preferably 42% or more, in subsequent pH 6.8 medium after 60 min.

19. Dosage form according to any of items 16 to 18, comprising a pharmaceutically, nutraceutically or cosmetically active ingredient containing core, coated with the composition according to any one of items 1 to 14 and with an active ingredient release of 10% or less after 2 hours in pH 1.2 medium and of 70 or more, preferably 88% or more in subsequent pH 6.8 medium after 120 min, preferably 90 min.

20. Dosage form according to any of items 16 to 19, comprising a pharmaceutically, nutraceutically or cosmetically active ingredient selected from prebiotics or probiotics.

21. Dosage form according to any of items 16 to 20, comprising a pharmaceutically, nutraceutically or cosmetically active ingredient selected from analgetics, antibiotics or anti-infectives, antibodies, antiepileptics, antigens from plants, antirheumatics, betablocker, benzimidazole derivatives, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptide hormones, proteins, therapeutical bacteria, peptides, urology drugs and vaccines.

22. Use of a composition according to any of items 1 to 14 as a coating or binding agent for pharmaceutically, nutraceutically or cosmetically active ingredients.

### EXAMPLES

### Preparation of Compositions

### Used Materials:

EUDRAGUARD® biotic dispersion is a 30 wt. % aqueous dispersion of (meth)acrylic copolymer containing 25 wt. % methyl methacrylate units, 65 wt. % methyl acrylate units, 10 wt. % methacrylic acid units.
Sodium stearate was purchased from Sigma-Aldrich.
Glyceryl tristearate (Dynasan®118) was purchased from Cremer.
Talc M was purchased from Imerys.
Fumed silica Aerosil200 with specific surface area BET=200 m2/g.
Triethyl citrate (TEC) was purchased from Merck.
Glycerol monostearate (GMS Imwitor 900 K) was purchased from Cremer.

### Example 1

Solution of 2.76 g sodium stearate in 99.04 g demineralized water heated to 70° ° C. was added to 80.0 g of EUDRAGUARD® biotic dispersion under stirring. A low viscous aqueous dispersion with pH=7.2 was formed. Talc M (12.0 g) was added to the resulting dispersion under stirring without additional heating, and stirring was continued for 5 minutes at 13000 rpm. The resulting suspension was sieved through 0.25 mm sieve and spray-dried using mini-spray-dryer Buchi B-290 (inlet temperature: 80° C., outlet temperature. 46-47° C.) to obtain a powder with a composition specified in Table 1.

### Example 2

Solution of 2.76 g sodium stearate in 60.64 g demineralized water heated to 70° C. was added to 80.0 g of EUDRAGUARD® biotic dispersion under stirring. A low viscous aqueous dispersion with pH>7.0 was formed. Glyceryl tristearate (Dynasan®118, 2.4 g) was added to the resulting dispersion under stirring without additional heating, and stirring was continued for 5 minutes at 13000 rpm. The resulting mixture was filtered through 0.25 mm sieve and spray-dried using mini-spray-dryer Büchi B-290 (inlet temperature: 80° C., outlet temperature. 46-47° C.) to obtain a powder with a composition specified in Table 1.

### Example 3

Solution of 8.23 g sodium stearate in 167.76 g demineralized water heated to 70° C. was added to 217.5 g of EUDRAGUARD® biotic dispersion under stirring. A low viscous aqueous dispersion with pH=7.2 was formed. Glyceryl tristearate (Dynasan®118, 6.52 g) was added to the resulting dispersion under stirring without additional heating, and stirring was continued for 20 minutes at 13000 rpm. The resulting mixture was filtered through 0.25 mm sieve and spray-dried using mini-spray-dryer Büchi B-290 (inlet temperature: 80° C., outlet temperature. 46-47° C.) to obtain a powder with a composition specified in Table 1.

### Comparative Example 1

Solution of 4.46 g sodium caprylate in 152.69 g demineralized water heated to 70° C. was added to 217.5 g of EUDRAGUARD® biotic dispersion under stirring. A low viscous aqueous dispersion with pH=6.7 was formed. Glyceryl tristearate (Dynasan®118, 6.52 g) was added to the resulting dispersion under stirring without additional heating, and stirring was continued for 15 minutes at 13000 rpm. The resulting mixture was filtered through 0.25 mm sieve and spray-dried using mini-spray-dryer Büchi B-290 (inlet temperature: 80° C., outlet temperature. 46-47° C.) to obtain a powder with a composition specified in Table 1.

### Comparative Example 2

Solution of 1.13 g triethyl citrate (TEC) in 87.02 g demineralized water heated to 70° C. was added to 75.0 g of EUDRAGUARD® biotic dispersion under stirring. A low viscous aqueous dispersion with pH>7.0 was formed. Talc M (11.25 g) was added to the resulting dispersion under stirring without additional heating, and stirring was continued for 5 minutes at 13000 rpm. The resulting mixture was filtered through 0.25 mm sieve and spray-dried using mini-spray-dryer Büchi B-290 (inlet temperature: 80° C., outlet temperature. 46-47° C.) to obtain a powder with a composition specified in Table 1.

### Comparative Example 3

Solution of 1.72 g sodium stearate in 37.88 g demineralized water heated to 70° C. was added to 50.0 g of EUDRAGUARD® biotic dispersion under stirring. A low viscous aqueous dispersion with pH>7.0 was formed. Fumed silica (Aerosil®200, 1.50 g) was added to the resulting dispersion under stirring without additional heating, and stirring was continued for 5 minutes at 13000 rpm. The resulting mixture was filtered through 0.25 mm sieve and spray-dried using mini-spray-dryer Büchi B-290 (inlet temperature: 80° C., outlet temperature. 46-47° C.) to obtain a powder with a composition specified in Table 1.

TABLE 1
Compositions used in examples and comparative examples
of the present invention with caffeine citrate pellets:

| | Polymer^{a} | Carboxylate, | Glidant, |
|---|---|---|---|
| Example | [wt. %] | (Amount) [wt. %] | (Amount) [wt. %] |
| Example 1 | 61.9 | Na-stearate (7.1) | talc M (31.0) |
| Example 2 | 82.3 | Na-stearate (9.5) | glyceryl |
| | | | tristearate (8.2) |
| Example 3 | 81.6 | Na-stearate (10.3) | glyceryl |
| | | | tristearate (8.2) |
| Comparative | 85.6 | Na-caprylate (5.9) | glyceryl |
| Example 1 | | | tristearate (8.6) |
| Comparative | 92.9 | Triethyl citrate (4.7) | talc M (46.5) |
| Example 2 | | | |
| Comparative | 82.3 | Na-stearate (9.5) | Aerosil ®200 (8.2) |
| Example 3 | | | |

^{a}polymer = (meth)acrylic copolymer containing 25 wt. % methyl methacrylate units, 65 wt. % methyl acrylate units, 10 wt. % methacrylic acid units.

### Examples 4-10 and Comparative Examples 4-9

Preparation of Coated Caffeine Citrate Pellets with Active Components

### Substrates

Caffeine citrate pellets contained 40.0 wt. % active pharmaceutical ingredient and had particle size of 707-1410 μm (No more than 10.0% can be retained in sieve #14 (ASTM) with mesh size 1410 μm; No more than 10.0% can pass through the sieve #25 (ASTM) with mesh size 707 μm). Diprophylline pellets contained 52.3 wt. % active pharmaceutical ingredient and had particle size of 800-1000 μm.

### Preparation of Coating Suspension

Redispersion of spray-dried powders was affected by adding the powder to demineralized water (powder/water=20 wt. %/80 wt. %) under stirring with an impeller stirrer at room temperature (ca. 22° C.). Stirring was continued for 1 hour.

### Preparation of Coated Diprophylline or Caffeine Citrate Pellets

100 g of diprophylline or caffeine citrate pellets were coated via spray-drying in a Hüttlin Mycrolab device using the dispersions prepared as described above. Table 1 summarizes the coating conditions for diprophylline pellets.

In example 10, the composition of example 3 was efficiently redispersed according to the procedure described above, but no coated pellet was prepared. In comparative example 4, the composition of comparative example 1 (with sodium caprylate instead of sodium stearate) could not be dispersed even after 1.5 hours of intensive stirring. This composition is not suitable for providing redispersible material.

TABLE 2
Conditions for coating of substrate pellets

| | |
|---|---|
| inlet temperature (° C.) | 29-40 |
| outlet temperature (° C.) | 23-28 |
| air flow rate (m³/h) | 14-29 m³/h |
| Nozzle bore (mm) | 0.8 |
| atomizing pressure (bar) | 0.8 |
| spray rate (g/min) | 0.5-6.5 |

The spraying time was 66-111 minutes depending on the amount of coating composition applied (4-15 wt. % relative to substrate mass). The coated pellets obtained by the spraying process were tested for release of active ingredient (diprophylline or caffeine citrate).

The dissolution (release) tests for coated pellets comprising active ingredients (diprophylline or caffeine citrate), were carried out using BP Method II paddle apparatus (Model PTWS, Pharmatest, Hainburg, Germany). The volume of the dissolution media was 900 mL maintained at 37±0.5° C. and a paddle speed of 100 rpm was employed. The amount of active ingredient released from the coated tablets or pellets was determined by UV spectrophotometer (271 nm for diprophylline). The pellets were first placed for 120 min into 0.1N HCl (pH=1.2), and subsequently into phosphate buffer with pH=6.8.

Table 3 summarizes the release tests of examples 4-9 and comparative examples 4-9. Release of <10% active component at pH=1.2 for 120 minutes shows that the respective coating is sufficiently gastro-resistant, release of more than 10% active ingredient indicates that coating composition is not suitable as a gastro-resistant coating. For efficient release in intestine, at least 70%, preferably 80%, more preferably 90% of active component release should be possible after 180 mins, preferably 90 minutes at the target pH level (6.8). As it can be seen from Table 3, both gastric resistance and efficient active component release at pH=6.8 are provided for compositions according to the invention. Particularly importantly, examples 5 and 6 show that compositions of the invention can be stored for a relatively long period of time (at least 3-6 months) and still remain suitable for synthesizing gastro-resistant coatings with efficient release at pH=6.8. Conversely, the composition of comparative example 2 (TEC instead of sodium stearate used) though acid-stable at particular amount of coatings applied, does not provide sufficient release at pH=6.8. Composition of comparative example 3 (Aerosil®200 used as a glidant) was not stable at pH=1.2.

TABLE 3
Release tests

| | | | Amount | release | release | release | release |
|---|---|---|---|---|---|---|---|
| | | | compo- | after 120 | after | after | after 90 |
| | | Active | sition | min, | 30 min, | 60 min, | min, |
| | | compo- | [wt. | pH = 1.2 | pH = 6.8 | pH = 6.8 | pH = 6.8 |
| Example | Composition | nent^{c} | %] | [%] | [%] | [%] | [%] |
| Ex. 4 | Ex. 1 | D | 15 | <10 | 53 | 100 | 100 |
| Ex. 5 | Ex. 1^{a} | D | 15 | <10 | 20 | 100 | 100 |
| Ex. 6 | Ex. 1^{b} | D | 15 | <10 | 65 | 100 | 100 |
| Ex. 7 | Ex. 2 | C | 6 | <5 | 20 | 70 | 95 |
| Ex. 8 | Ex. 2 | C | 8 | <5 | <5 | 45 | 90 |
| Ex. 9 | Ex. 2 | D | 15 | <10 | 50 | 100 | 100 |
| Comp. Ex. 4 | Comp. Ex. 2 | D | 15 | <5 | <5 | <5 | 40 |
| Comp. Ex. 5 | Comp. Ex. 2 | C | 6 | <5 | <5 | 40 | 80 |
| Comp. Ex. 6 | Comp. Ex. 3 | C | 4 | 90 | 100 | 100 | 100 |
| Comp. Ex. 7 | Comp. Ex. 3 | C | 6 | 58 | 75 | 86 | 96 |
| Comp. Ex. 8 | Comp. Ex. 3 | C | 8 | 15 | 25 | 38 | 50 |
| Comp. Ex. 9 | Comp. Ex. 3 | C | 10 | <10 | 10 | 26 | 47 |

Ex. No = example according to invention;
Comp. Ex. No = comparative example.
^{a}after 3 months' storage;
^{b}after 6 months' storage;
^{c}D = diprophylline; C = caffeine citrate

### Preparation for Caffeine Microcrystalline Cellulose Core Pellets

### Preparation of Compositions

### Used Materials:

EUDRAGUARD® biotic dispersion is a 30 wt. % aqueous dispersion of (meth)acrylic copolymer containing 25 wt. % methyl methacrylate units, 65 wt. % methyl acrylate units, 10 wt. % methacrylic acid units. The below stated amounts in the following examples and comparative examples refer to the amount of the dry weight of the (meth)acrylic copolymer not the total weight of the EUDRAGUARD® biotic dispersion. For example, if it is described that 1% of the total composition EUDRAGUARD® biotic is contained, it means that 1% of the (meth)acrylic copolymer, which makes up only 30 wt.-% of EUDRAGUARD® biotic is used, i.e. 3.33% of the commercially available EUDRAGUARD® biotic aqueous dispersion are used.
Caffeine purchased from Aarti Drugs Ltd.
Microcrystalline cellulose PH 101 purchased from JRS Pharms
Vivapur MCG 611P a microcrystalline cellulose purchased from JRS Pharma
Sodium stearate purchased from Tokyo Chemical Indrustry Co., Ltd
Triethyl citrate purchased from Vertellus
Guar gum purchased from Polygal AG
Talc purchased from IMERYS
NU-FLOW® purchased from RIBUS Inc.
NU-MAG® purchased from RIBUS Inc.
HPMC (5 cps) purchased from JRS Pharma
Glyceryl monostearate purchased from IOI Oeo
Magnesium stearate purchased from Prachin Chemical
Corn starch purchased from Universal Starch Chem Allied Ltd.

### Caffeine Microcrystalline Cellulose Core Pellets Contained

| Ingredients | % w/w |
|---|---|
| Caffeine | 35.0 |
| Microcrystalline cellulose PH 101 | 50.0 |
| Vivapur MCG 611P | 25.0 |
| Purified water | q.s. |
| Total | 100.0 |

### Process for Preparation of Caffeine Microcrystalline Cellulose Core Pellets

I. Caffeine, microcrystalline cellulose and Vivapur MCG 611P was sifted using #30 ASTM
II. Sifted blend was mixed in rapid mixture granulator for 10 minute and granulated with purified water.
III. Granulated blend of step-II was used for extrusion and followed by spheronization using Fuji Paudal spheronizer

Extrusion parameters

| Extrusion type | Cone |
|---|---|
| Extrusion screw | Single |
| Screw speed (rpm) | 40-60 |
| Screen aperture diameter(mm) | 1 |
| Extrusion pressure (bar) | 2-2.5 |
| Feed rate (g/min.) | 100-200 |
| Extrusion temperature(° C.) | 25-30 |

Spheronization parameters

| | |
|---|---|
| Spheronization plate type | Notched circular |
| Spheronization plate size(mm) | 2.0 |
| Spheronization speed (rpm) | 1500-1700 |
| Spheronization load (g) | 400-600 |

### Drying and Sizing of the Active Containing Core Pellets

I. All spheronised pellets were dried in fluid bed processor at 60 ºC inlet temperature until final LOD of the pellets were less than 5% w/w.
II. Dried pellets were sized using sieve shaker to have the pellets fraction of 14/25 ASTM mesh.
III. 14/25 ASTM mesh pellets fraction was used for the coating.

| Ingredients | Comp Ex. 10 | | Comp Ex. 11 | Comp Ex 12 |
|---|---|---|---|---|
| Caffeine pellets | 89.97 | 81.77 | 87.99 | 88.59 |
| EUDRAGUARD ® biotic | 9.0 | 16.35 | 8.80 | 11.07 |
| Sodium stearate | 1.03 | 1.88 | 1.01 | — |
| Triethyl citrate | — | — | — | — |
| Glyceryl monostearate | | | | 0.34 |
| Guar Gum | — | — | 2.20 | — |
| Purified water | q.s* | | q.s* | q.s.* |
| Total | 100.0 | | 100.0 | 100.0 |
| % w/ w solids in | 12 | | 8 | 12 |
| dispersion | | | | |
| Polymeric coating | 10 | 20 | 10 | 12.5 |
| level (%) | | | | |

*Quantity sufficient to make the volume.

| | % Composition | | | | | | | | | | |
|---|---|---|---|---|---|---|---|---|---|---|---|
| Ingredients | Ex. 11 | Ex. 12 | Ex. 13 | Ex. 14 | | Ex. 15 | | Ex. 16 | | Ex. 17 | |
| Caffeine pellets | 88.56 | 88.25 | 86.10 | 88.25 | 85.73 | 91.17 | 86.10 | 88.25 | 85.73 | 90.81 | 88.77 |
| EUDRAGUARD ® biotic | 7.08 | 7.06 | 8.61 | 7.06 | 8.57 | 5.66 | 8.61 | 7.06 | 8.57 | 7.26 | 8.88 |
| Sodium stearate | 0.81 | 0.81 | 0.99 | 0.81 | 0.99 | 0.63 | 0.99 | 0.81 | 0.99 | 0.84 | 1.02 |
| Triethyl citrate | — | 0.35 | — | 0.35 | 0.43 | — | — | 0.35 | 0.43 | 0.36 | 0.44 |
| Talc | 3.54 | 3.53 | — | — | — | — | — | — | — | — | — |
| NU-FLOW ® | — | — | 4.30 | 3.53 | 4.29 | — | — | — | — | — | — |
| NU-MAG ® | — | — | — | — | — | 2.73 | 4.30 | 3.53 | 4.29 | — | — |
| HPMC | — | — | — | — | — | — | — | — | — | 0.89 | 0.89 |
| Purified water | q.s* | — | q.s* | q.s* | — | q.s* | — | q.s* | — | q.s* | — |
| Total | 100 | — | 100 | 100 | — | 100 | — | 100 | — | 100 | — |
| % w/w solids in | 12 | — | 12 | 12 | — | 12 | — | 12 | — | 12 | — |
| dispersion | | | | | | | | | | | |
| Polymeric coating level | 8 | 8 | 10 | 8 | 10 | 8 | 10 | 8 | 10 | 8 | 10 |
| (%) | | | | | | | | | | | |

*Quantity sufficient to make the volume.

| | % Composition | | | | | | | | | |
|---|---|---|---|---|---|---|---|---|---|---|
| Ingredients | Ex. 18 | | Ex. 19 | | Ex. 20 | Ex.20a | Ex. 21 | Ex 22 | Ex 23 | Ex 24 |
| Caffeine pellets | 88.56 | 86.10 | 88.25 | 85.73 | 76.70 | 86.81 | 81.10 | 71.24 | 71.24 | 70.61 |
| EUDRAGUARD ® biotic | 7.41 | 8.61 | 7.06 | 8.57 | 19.18 | 10.85 | 16.22 | 17.81 | 17.81 | 17.65 |
| Sodium stearate | 0.81 | 0.99 | 0.81 | 0.99 | 2.21 | 1.25 | 1.87 | 2.05 | 2.05 | 2.03 |
| Triethyl citrate | — | — | 0.35 | 0.43 | — | — | — | — | — | 0.88 |
| Glyceryl behenate | — | — | — | — | 1.92 | 1.09 | — | — | — | — |
| Glyceryl monostearate | — | — | — | — | — | — | 0.81 | — | — | — |
| Magnesium stearate | 3.54 | 4.30 | 3.53 | 4.29 | — | — | — | — | — | — |
| Microcrystalline cellulose (PH101) | — | — | — | — | — | — | — | 8.9 | — | — |
| Corn starch | — | — | — | — | — | — | — | — | 8.9 | 8.83 |
| Purified water | q.s* | — | q.s* | — | q.s* | q.s* | q.s* | q.s* | q.s* | q.s* |
| Total | 100 | — | 100 | — | 100 | 100 | 100 | 100 | 100 | 100 |
| % w/w solids in dispersion | 12 | — | 12 | — | 12 | 12 | 12 | 12 | 12 | 12 |
| Polymeric coating level (%) | 8 | 10 | 8 | 12.510 | 25 | 12.5 | 25 | 25 | 25 | 25 |

*Quantity sufficient to make the volume.

### Preparation of Coating Dispersion of Experiments and Comparative Experiments

Preparation of Coating Dispersion for Pellets Coating: Comp. Ex. 10
I. The hot purified water (˜ 70° C.) under stirring. to it adds sodium stearate until it forms clear solution
II. EUDRAGUARD® biotic under overhead stirrer 1000-1200 rpm step-1 solution added dispersion becomes viscous stir for about 30 minutes.
III. The coating dispersion was passed through 40 #ASTM sieve (425 μm) and used for coating of tablets as described in the next section.

### Preparation of Coating Dispersion for Pellets Coating: Comp Ex. 11, Ex. 11, 13, 15, 18, 20, 21, 22, and 23

I. The hot purified water (˜70° C.) under stirring. to it adds sodium stearate until it forms clear solution
II. EUDRAGUARD® biotic under overhead stirrer 1000-1200 rpm step-1 solution added dispersion becomes viscous stir for about 30 minutes.
III. Added Talc, NU-FLOW®, NU-MAG®, magnesium stearate, guar gum, glyceryl behenate, or glyceryl monostearate to the dispersion of step-2 under stirring. Continued stirring for another 20 minutes
IV. The coating dispersion was passed through 40 #ASTM sieve (425 μm) and used for coating of pellets as described in the next section.

### Preparation of Coating Dispersion for Pellets Coating: Ex. 12, 14, 16, 17, 19, and 24

V. The hot purified water (˜70° C.) under stirring. to it adds sodium stearate until it forms clear solution
VI. EUDRAGUARD® biotic under overhead stirrer 1000-1200 rpm step-1 solution added dispersion becomes viscous stir for about 30 minutes.
VII. Add triethyl citrate in the step 2 dispersion and stir dispersion
VIII. Add Talc, NU-FLOW®, NU-MAG®, HPMC 5 cps, or magnesium stearate, added to the dispersion of step-2 under stirring. Continued stirring for another 20 minutes
IX. The coating dispersion was passed through 40 #ASTM sieve (425 μm) and used for coating of pellets as described in the next section.
   Preparation of Coating Dispersion for Pellets Coating: Comparative Example 12 is Similar to Example 4 in U.S. Pat. No. 5,644,011 Using Eudraguard® Biotic
I. Eudraguard biotic mixed with glyceryl monostearate emulsion in water (Glyceryl monostearate emulsion prepared by dispersing it in hot water (70° C.) under high shear homogenization).

### Coating of Pellets

Caffeine drug loaded pellets coated with coating dispersions described in previous section using a fluid bed processor (GPCG 1.1) with following parameters

| Batch information/ | | Comp | Comp | Comp |
|---|---|---|---|---|
| Process parameter | Unit | Ex. 10 | Ex. 11 | Ex. 12 |
| Batch size | g | 600 | | 390 |
| Atomization air | Bar | 1-1.2 | | 1.1 |
| pressure | | | | |
| Inlet temperature | ° C. | 26-28 | 40-45 | 33-35 |
| Bed temperature | ° C. | 22-25 | 35-37 | 26-29 |
| Air flow | CFM | 65-75 | 70-90 | 55-69 |
| Spray rate range | g/min | 4 (1-5) | 5.46(1-7) | 2-5 |
| Process observation | | Good | Slow | Good |
| | | process | process | process |

| Batch information/ | | | | | | | | |
|---|---|---|---|---|---|---|---|---|
| Process | | | | | | | | |
| parameter | Unit | Ex. 11 | Ex. 12 | Ex. 13 | Ex. 14 | Ex. 15 | Ex. 16 | Ex. 17 |
| Batch size | g | 600 | | | | | | |
| Atomization air | Bar | 1-1.2 | | | | | | |
| pressure | | | | | | | | |
| Inlet temperature | 0° C. | 28-35 | 28-30 | 26-28 | 28-32 | 28-30 | 28-30 | 30-40 |
| Bed temperature | 0° C. | 24-30 | 23-25 | 21-24 | 24-26 | 23-26 | 21-24 | 29-32 |
| Air flow | CFM | 60-70 | 50-60 | 60-70 | 50-60 | 60-70 | 60-70 | 60-70 |
| Spray rate range | g/min | 4.6(1-8) | 4.6(1-8) | 5.92(1-8) | 8(3-11) | 7.33(4-12) | 8(4-10) | 6(3-7) |
| Process | | Good process | | | | | | |
| observation | | | | | | | | |

| Batch information/ | | | | | | | | | |
|---|---|---|---|---|---|---|---|---|---|
| Process parameter | Unit | Ex. 18 | Ex. 19 | Ex. 20 | Ex. 21 | Ex. 22 | Ex. 23 | Ex. 24 | Ex.20a |
| Batch size | g | 600 | | | | | | | 400 |
| Atomization air | Bar | 1-1.2 | | | | | | | 1.1 |
| pressure | | | | | | | | | |
| Inlet temperature | 0° C. | 29-30 | 28-30 | 28-30 | 29-30 | 28-30 | 28-30 | 28-30 | 33-35 |
| Bed temperature | 0° C. | 23-26 | 24-28 | 22-26 | 23-26 | 22-27 | 23-27 | 23-26 | 26-29 |
| Air flow | CFM | 60-70 | 60-75 | 70-80 | 44-55 | 60-75 | 65-75 | 50-60 | 55-69 |
| Spray rate range | g/min | 6.42(1-8) | 7(4-10) | 6.5(1-8) | 6(1-7) | 6(5-10) | 6(5-8) | 5.5(1-7) | 3-7.5 |

| | |
|---|---|
| Process observation | Good process |

### Evaluation of Coated Pellets

Coated pellets were evaluated for surface appearance and enteric dissolution in 0.1N HCl for 120 minutes followed by pH 6.8 phosphate buffer for 180 minutes

| Parameter | Comp Ex. 10 | | Comp Ex. 11 | | Comp Ex. 12 |
|---|---|---|---|---|---|
| surface texture/ | Off white | | Off white | | Off white |
| color/feel | smooth | | smooth | | smooth |
| Coating levels | 8% | 10% | 8% | 10% | 12.5% |
| Dissolution | | | | | |
| profile | | | | | |
| Acid Stage | 18 | 12 | 100 | 100 | 2.8 |
| 0.1N N HCl for | | | | | |
| 120 min: not | | | | | |
| more than 10% | | | | | |
| Buffer stage | — | — | — | — | 3.2 |
| pH 6.8 phosphate | | | | | |
| buffer for 165 min | | | | | |
| Buffer stage | 91 | 84 | 100 | 100 | 3.3 |
| pH 6.8 Phosphate | | | | | |
| buffer for 180 min: | | | | | |
| not less than 70% | | | | | |

| | | |
|---|---|---|
| Remark | Impaired acid protection | No Release |
| | | in pH 6.8 |

| Parameter | Ex. 11 | | Ex. 12 | | Ex. 13 | | Ex. 14 | | Ex. 15 | | Ex. 16 | |
|---|---|---|---|---|---|---|---|---|---|---|---|---|
| surface texture/color/feel | Off white smooth | | | | Brownish rough | | | | Slight yellowish smooth | | | |
| Coating levels | 8% | | 8% | | 10% | | 8% | 10% | 8% | 10% | 8% | 10% |
| Dissolution profile | | | | | | | | | | | | |
| Acid Stage | 9 | | 8 | | 7 | | 5 | 3 | 3 | 2 | 1 | 0 |
| 0.1N N HCl for | | | | | | | | | | | | |
| 120 min: | | | | | | | | | | | | |
| not more than 10% | | | | | | | | | | | | |
| Buffer stage | 88 | | 92 | | 86 | | 93 | 89 | 87 | 83 | 85 | 81 |
| pH 6.8 Phosphate buffer for | | | | | | | | | | | | |
| 180 min: not less than 70% | | | | | | | | | | | | |
| Remark | Good acid protection & acceptable buffer release | | | | | | | | | | | |
| Parameter | Ex. 17 | | Ex. 18 | | Ex. 19 | | Ex. 20 | Ex. 21 | Ex. 22 | Ex. 23 | Ex. 24 | Ex. 20a |
| surface texture/ | Off white | | White smooth | | | | White | White | Off | Off | Off | White |
| color/feel | smooth | | | | | | smooth | smooth | white | white | white | smooth |
| | | | | | | | | | rough | smooth | smooth | |
| Coating levels | 8% | 10% | 8% | 10% | 8% | 10% | 25% | 25% | 25% | 25% | 25% | 12.5% |
| dissolution profile | | | | | | | | | | | | |
| Acid Stage | 2 | 1 | 7 | 6 | 5 | 4 | 2 | 8 | 3 | 3 | 7 | 4.3 |
| 0.1N N HCl for | | | | | | | | | | | | |
| 120 min: | | | | | | | | | | | | |
| not more than | | | | | | | | | | | | |
| 10% | | | | | | | | | | | | |
| Buffer stage pH | — | — | — | — | — | — | — | — | — | — | — | 51.7 |
| 6.8 phosphate | | | | | | | | | | | | |
| buffer for 165 | | | | | | | | | | | | |
| min | | | | | | | | | | | | |
| Buffer stage | 83 | 75 | 85 | 83 | 80 | 76 | 100 | 73 | 100 | 100 | 75 | 76.5 |
| pH 6.8 | | | | | | | | | | | | |
| Phosphate buffer | | | | | | | | | | | | |
| for 180 min: | | | | | | | | | | | | |
| not less than | | | | | | | | | | | | |
| 70% | | | | | | | | | | | | |

| | |
|---|---|
| Remark | Good acid protection and acceptable buffer release |

1. Composition, suitable as coating or binding agent for pharmaceutically, nutraceutically or cosmetically active ingredients, comprising the following components:
a) at least one (meth)acrylate copolymer a), comprising polymerized units of 5 to 25% by weight of methacrylic acid and 75 to 95% by weight of C1- to C4-alkylesters of methacrylic acid and/or C1- to C4-alkylesters of acrylic acid, and
b) 1 to 25% by weight, based on the total weight of at least one (meth)acrylate copolymer a), of an alkali or ammonium salt of a saturated aliphatic monocarboxylic acid having 10 to 30 carbon atoms,
and c) or d) or both c) and d)
c) 2 to 25% by weight, based on the total weight of a), of at least one compound selected from glyceryl tristearate, hydroxypropyl methylcellulose, glyceryl behenate, and glyceryl monostearate and/or
d) 25 to 90% by weight, based on the total weight of a), of at least one compound selected from talc, rice hulls in powder form, magnesium stearate, corn starch, and microcrystalline cellulose.

2. Composition according to claim 1, wherein the at least one (meth)acrylate copolymer a) comprises an overall monomer composition by weight comprising polymerized units of 10 to 30% by weight methyl methacrylate, 50 to 70% by weight methyl acrylate and 5 to 15% by weight methacrylic acid.

3. Composition according to claim 1, wherein the alkali or ammonium salt of a saturated aliphatic monocarboxylic acid having 10 to 30 carbon atoms is selected from alkali or ammonium salts of decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid and triacontanoic acid (melissic acid, C30).

4. Composition according to claim 1, wherein the alkali salt of the saturated aliphatic monocarboxylic acid is sodium stearate.

5. Composition according to claim 1, wherein up to 400% by weight, based on the total weight of the at least one (meth)acrylate copolymer a), of additional pharmaceutical, nutraceutical or cosmetic excipients are comprised.

6. Composition according to claim 5, wherein pharmaceutical, nutraceutical or cosmetic excipients are selected from the classes of antioxidants, brighteners, flavouring agents, flow aids, fragrances, penetration-promoting agents, pigments, plasticizers, polymers, pore-forming agents or stabilizers or combinations thereof.

7. Composition according to claim 1, wherein a plasticizer is present in an amount of 2 to 40% by weight, based on the total weight of the at least one methacrylate copolymer a).

8. Composition according to claim 1, wherein a plasticizer is present which is selected from the groups of alkyl citrates, glycerol esters, alkyl phthalates, alkyl sebacates, sucrose esters, sorbitan esters and polyethylene glycols.

9. Composition according to claim 1, wherein a plasticizer is present which is selected from triethyl citrate (TEC), acetyl triethyl citrate (ATEC), diethyl sebacate and dibutyl sebacate (DBS), glycerol, propylene glycol, polyethylene glycols 200 to 12,000 and castor oil.

10. Composition according to claim 1, in the form of an aqueous dispersion.

11. Composition according to claim 1, in the form of a re-dispersible powder.

12. Composition according to claim 1, comprising
a) the at least one (meth)acrylate copolymer a);
b) 1 to 25% by weight, based on the total weight of a), of sodium stearate,
and c) or d) or both
c) 5 to 15% by weight, based on a), hydroxypropyl methylcellulose and/or glyceryl tristearate;
d) 40 to 60% by weight, based on a), of talc and/or magnesium stearate;
wherein the components a), b), c) and d) add up to 100% dry weight.

13. Aqueous dispersion, comprising water and 5 to 50% by weight of the composition according to claim 1.

14. Dosage form, comprising a pharmaceutically, nutraceutically or cosmetically active ingredient and a polymeric coating or a polymeric matrix, wherein the polymeric coating or the polymeric matrix comprises the composition according to claim 1.

15. Use-A method of using a composition according to claim 1, comprising providing the composition as a coating or binding agent for pharmaceutically, nutraceutically or cosmetically active ingredients.

16. Composition according to claim 2, wherein the alkali or ammonium salt of a saturated aliphatic monocarboxylic acid having 10 to 30 carbon atoms is selected from alkali or ammonium salts of decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid and triacontanoic acid (melissic acid, C30).
